(19) United States
(12) Patent Application Publication
(10) Pub. No.: US 2005/0260264 A1
(43) Pub. Date: Nov. 24, 2005

(54) Dosage form for delivery of multiple drug forms

(76) Inventors: David E. Edgren, Los Altos, CA (US); Bih-Hsiung Hsu, San Jose, CA (US); Shu Li, Union City, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/133,769

(22) Filed: May 20, 2005

Related U.S. Application Data
(60) Provisional application No. 60/573,312, filed on May 21, 2004.

(51) Int. Cl. A61K 9/22; A61K 9/24

(52) U.S. Cl. 424/468

(57) ABSTRACT

Disclosed are controlled release dosage forms and related methods including: (a) a micronized or liquid base form of a drug; (b) either a pharmaceutically acceptable salt form of the drug or starting materials that are capable of reacting to form a pharmaceutically acceptable salt form of the drug; (c) an upper gastrointestinal system pharmaceutically acceptable salt form releasing structure; and (d) a colonic system base form releasing structure.

### FIELD OF THE INVENTION

The invention pertains to the controlled delivery of pharmaceutical agents, dosage forms and methods thereof. In particular, the invention is directed to dosage forms and methods for delivering multiple drug forms to achieve a therapeutic effect. Specifically, the invention relates to a method of administering differing drug forms at different regions of the gastrointestinal tract, each drug form being delivered at a separately controlled or controllable release rate over a sustained period.

### BACKGROUND OF THE INVENTION

In conventional pharmaceutical development the choice of dosage forms such as an acid, base or salt is based on obtaining, on the one hand, the most stable dosage form, and on the other, to provide maximum absorption in the upper GI tract. As most drug dosage forms are designed for immediate release of the drug dosage, the dosage form is made to be well dissolved in the upper GI tract and usually highly dissociated, i.e., highly charged, in the GI environment of the small and large intestines (pH=approximately 5-7).

Pharmaceutical development also typically targets drug forms for absorption in the upper GI instead of the lower GI because the upper GI tract has a far greater surface area for absorption of drugs than does the lower GI tract. Since the colon does not have the microvilli of the small intestine, the ratio of absorption surface of the small intestine to that of the colon is 480.

However, some drug forms that manifest high absorption in the upper GI have very poor absorption in the lower GI. For example, certain drug salts of high solubility are indeed absorbed well in the small intestine but not well absorbed in the lower GI tract. Also, drug compositions comprising amino acids are typically absorbed in the GI tract via amino acid transporters. Since the amino acid transporters are found almost exclusively in the small intestine (and not in the colon), absorption of many amino acid drug compositions is much poorer in the colon than in the small intestine.

Since the typical residence time of a drug in the upper GI tract is from approximately four to six hours, drugs having poor colonic absorption are absorbed by the body through a period of only four to six hours after oral ingestion. Frequently it is medically desirable that the administered drug be presented in the patient's blood stream at a relatively constant concentration throughout the day. To achieve this with traditional drug formulations that exhibit minimal colonic absorption, patients would need to ingest the drugs three to four times a day. Practical experience with this inconvenience to patients suggests that his is not an optimum treatment protocol. Accordingly, it is desired that a once daily administration of such drugs, with long-term absorption throughout the day, be achieved.

To provide constant dosing treatments, conventional pharmaceutical development has suggested various controlled release drug systems. Such systems function by releasing their payload of drugs over an extended period of time following administration. However, these conventional forms of controlled release systems are less effective in the case of drugs exhibiting minimal colonic absorption. Since the drugs are only absorbed in the upper GI tract and since the residence time of the drug in the upper GI tract is only 4 to 6 hours, the fact that a proposed controlled release dosage form may release its payload after the residence period of the dosage form in the upper GI does not mean the that body will continue to absorb the controlled release drug past the 4 to 6 hours of upper GI residence. Instead, the drug released by the controlled release dosage form after the dosage form has entered the lower GI tract is generally not absorbed and is expelled from the body with other matter from the lower GI.

In response to and in recognition of this, the prior art has attempted to provide a remedy by fashioning mechanisms intended to increase the residence time in the upper GI tract of extended release drug dosage forms. These have usually provided only marginally improved results.

More recently, U.S. Pat. No. 6,419,954 disclosed a tablet in which release of active agent is controlled by use of multiple bioerodible layers of different active agents, different amounts of active agents and/or different forms of active agents. As the multi-layered tablet slowly dissolves in its passage through the digestive tract, it releases varying amounts of active agent or different active agents at different times, and in different anatomical compartments. However, this patent does not disclose how to address the problems of differential absorption of drugs between the upper and lower GI.

Thus, there is a need to develop compounds, methods and products to improve absorption of drugs throughout the GI tract. The benefits of such compounds, methods and products are numerous.

### SUMMARY OF THE INVENTION

In an aspect, the invention relates to a controlled release dosage form comprising (a) a micronized or liquid base form of a drug; (b) either (i) a pharmaceutically acceptable salt form of the drug or (ii) starting materials that are capable of reacting to form a pharmaceutically acceptable salt form of the drug; (c) an upper gastrointestinal system pharmaceutically acceptable salt form releasing structure; and (d) a colonic system base form releasing structure. In another aspect, the invention relates to the above controlled release dosage form, wherein the upper gastrointestinal system pharmaceutically acceptable salt form releasing structure and the colonic system base form releasing structure are substantially coincident. In still another aspect, the invention relate to the above controlled release dosage form, wherein the upper gastrointestinal system pharmaceutically acceptable salt form releasing structure and the colonic system base form releasing structure are substantially separate.

The invention further relates to the above controlled release dosage form, wherein the controlled release dosage form comprises an osmotic controlled release dosage form. Additionally, the invention relates to the above controlled release dosage form, wherein the controlled release dosage form comprises a pharmaceutically acceptable salt of the drug. The invention relates to the above controlled release dosage form wherein the starting materials that are capable of reacting to form a pharmaceutically acceptable salt form of the drug comprise a salt forming agent and a micronized or a liquid base form of the drug. The invention additionally relates to the above controlled release dosage form, comprising an ion exchange layer; a drug layer; and a push layer. Further, the invention relates to the above controlled release dosage form, comprising a pharmaceutically acceptable salt of the drug.

### BRIEF DESCRIPTION OF THE DRAWINGS

FIGS. 1A through 1J show illustrative diagrams of various delivery profiles that can be attained by sequential delivery of a salt and a base form of a drug according to the present invention.

FIG. 2 shows an example of a sequential drug delivery system or dosage form according to one embodiment of the invention in which the salt and non-salt forms of a drug are provided in a mixture;

FIG. 3 shows an example of a sequential drug delivery system or dosage form according to one embodiment of the invention in which the drug forms are delivered from separate layers;

FIG. 4 shows an example of a sequential drug delivery system or dosage form similar to the embodiment of FIG. 4, further comprising an ion-exchange layer.

FIG. 5 shows an example of a sequential drug delivery system or dosage form similar to the embodiment of FIG. 4, further comprising an ion-exchange channel.

### DETAILED DESCRIPTION OF THE INVENTION

The invention will now be further described by reference to the following detailed description, drawings and examples.

All documents referred to herein are incorporated by reference as if reproduced fully herein.

Overview:

The inventors have unexpectedly discovered that it is possible to optimize drug over the length of the GI tract by delivering a drug in different ionization states at different locations along the GI tract. Use of drugs having different ionization states enables enhanced absorption over the course of delivery from the upper GI.

This takes advantage of the fact that drug absorption may be expressed, in part, as the product of drug permeability and drug solubility. See Philip S. Burton et al., Predicting Drug Absorption: How Nature Made It a Difficult Problem, J. of Pharmacology and Experimental Ther., Vol. 303:3 (2002). While there are many additional factors that govern drug absorption, such as the influence of GI transporters, these are two of the major factors. Accordingly, it is possible to select certain drugs and deliver them at different ionization states, dictated by ambient pH, that optimizes absorption.

For instance, a drug may exist in base form at the higher pH ranges present in the colon. While the base form may be less soluble (as measured at neutral pH), it is more permeable; meaning that the base form can pass through the colon epithelium more easily than a charged species. The same drug, if present in the lower pH regions of the upper GI tract, may be present as a pharmaceutically acceptable salt of the drug. This implies higher solubility, but lower permeability through the lipid membranes of the GI epithelium. While drugs are absorbed across the GI tract by both active and passive mechanisms, passive diffusion remains an important component of overall absorption. In passive diffusion, the driving force for absorption is the product of the drug concentration at the epithelial wall and the epithelial wall permeability. Therefore, by presenting the soluble salt form in the upper GI tract where the permeability is low followed by presenting the base form in the lower GI tract where the solubility is low and the permeability is high, the net effect is to provide a more complete and even absorption of drug than could be achieved by dosing either form singly.

The inventors have adapted this understanding to controlled release dosage forms designed to release drugs having different ionization states at different locations in the GI tract. This approach therefore improves oral bio-availability and efficacy of such drugs. The present delivery system thus significantly improves the therapeutic value of many drugs by delivering them in this sequential mode. The above description of sequentially delivered drug forms is simply by way of illustration. The invention provides a delivery system or dosage form that can be adapted to deliver two or more drug forms either simultaneously or sequentially.

Definitions:

As used herein:

“aqueous environment” means surroundings that contain liquid water.

“colon” or “colonic” refers to the large. colon.

“colonic system base form releasing structure” means a releasing structure that operates to release a micronized or a liquid base form of a drug in the colon. These structures can be constructed to operate based on estimates of where the inventive dosage form might be located at a given time following dosing to a patient. For instance, if the typical colonic transit time window for a given dosage form begins an average of around 8 hours following dosing and continues until an average of around 12 hours following dosing, then the colonic system base form releasing structure can be designed to release the base form within the average 8-12 hour window following dosing. The methods of such design are generally conventionally known.

“controlled release” means continuous release of a pharmaceutical agent over a prolonged period of time, wherein the pharmaceutical agent is released at a controlled rate over a controlled period of time.

“dosage form” means a drug composition or device capable of delivering a pharmaceutical agent. Suitable examples of dosage forms include, but are not limited to tablets, capsules, gel-caps, matrix forms, osmotic forms, immediate release forms, controlled release forms, sustained release forms, extended release forms, and the like. Other useful approaches for obtaining the inventive dosage form include diffusion systems such as matrix devices, dissolution systems such as encapsulated dissolution systems, combination diffusion/dissolution systems, and ion exchange resin systems such as described in “Remington's Pharmaceutical Sciences”, 1990 ed., pp. 1682-1685. Specific examples of the above approaches include, erodible matrix tablets, tiny pills, drug releasing beads, and hybrids of these and others.

“drug”, “pharmaceutical agent,” “active agent”, or “therapeutic agent” means an agent, drug, or compound having therapeutic characteristics or a pharmaceutically-acceptable acid addition salt, prodrug, or derivative thereof. “Base form” refers to a base of the drug, also known as a free base of the drug. “Form of a drug” refers to the state of the drug, particularly the ionization state of the drug, such as the base form or the salt form. Drugs may be incorporated into the drug composition and/or dosage forms of the present invention in amounts in the range of from about 1 milligram to about 750 milligrams, preferably in the range of from about 5 mg to about 250 mg, more preferably in the range of from about 10 mg to about 250 mg.

Generally, any drug that can exist in both a base form and a salt form is useful in the practice of this invention. Illustrative examples of base/salt drug combinations that can be delivered by the inventive dosage forms include, but are not limited to: Bupropion base and its HCl salt; HCl Chlordiaepoxide base and its HCl salt; Cimetidine base and its HCl salt; ciprofloxacin base and its HCl salt; Clindamycin base and its HCl salt; Codeine base and its phosphate salt; Fexofenadine base and its HCl salt; Fluphenazine base and its HCl salt; Hydromorphone base and its HCl salt; Hydrocodone base and its tartrate salt; Metformin base and its HCl salt; Minocycline base and its HCl salt; Nicardipine base and its HCl salt; Ondansetron base and its HCl salt; Oxycodone base and its HCl salt; and Tramadol base and its HCl salt.

“exit” and “exit orifice” means an opening in a dosage form which permits drug to exit the dosage form. Suitable examples are described below in more detail.

“immediate-release dosage form” refers to a dosage form that releases greater than or equal to about 80% of the pharmaceutical agent in less than or equal to about 1 hour.

“low solubility” means that the neat pharmaceutical agent (in the absence of surfactants or other excipients) exhibits a solubility of less than about 100 mg/ml in de-ionized water at 37° C. Preferably, low solubility shall mean a solubility of less than about 50 mg/ml, more preferably, less than about 25 mg/ml, more preferably still, less than about 15 mg/ml, more preferably still, less than about 10 mg/ml, more preferably still, less than about 5 mg/ml, most preferably, less than about 1 mg/ml.

As defined herein, the solubility of a pharmaceutical agent is determined by adding the pharmaceutical agent to stirred or agitated de-ionized water maintained in a constant temperature bath at a temperature of 37° C. until no more pharmaceutical agent dissolves. The resulting solution saturated with the pharmaceutical agent is then filtered, typically under pressure through a 0.8-micron Millipore filter, and the concentration of the pharmaceutical agent in the solution is measured by any appropriate analytical method including gravimetric, ultraviolet spectrophometry, chromatography, and the like. The solubility of the pharmaceutical agent is measured at saturated concentration.

“liquid” means a fluid form. In particular, base forms of drugs according to the present invention may be in the form of a liquid base form. Examples of liquid base forms of drugs useful in the practice of this invention, together with pharmaceutically acceptable salts thereof, include but are not limited to amphetamine base/amphetamine sulfate; bropheniramine base/bropheniramine maleate; and carbinoxamine base/carbinoxamine maleate.

“micronized” means to reduce to particles that possess an average diameter of less than about 30 microns in diameter, preferably less than about 20 microns, more preferably less than about 10 microns, and still more preferably less than about 5 microns. A micronized drug form promotes better and more uniform absorption than forms having a larger average size and a wider distribution of particle sizes. Drugs according to the invention can be purchased in micronized form or can be micronized using conventional micronization equipment, such as the Micron-Master™ line of micronizers available from The Jet Pulverizer Company (Moorestown, N.J.), or processed by a third-party micronization processor such as Micron Technologies (Exton, Pa.).

“non-drug salt” means a pharmaceutically acceptable salt of a compound other than the drug. Such compound may be pharmaceutically active. An example of a non-drug salt includes, but is not limited to sodium chloride or magnesium chloride.

“pharmaceutically acceptable salt”, or “salt form”, unless otherwise noted herein, shall mean any salt whose anion or cation does not contribute significantly to the toxicity or pharmacological activity of the salt, and, as such, they are the pharmacological equivalents of the base of the compound. Suitable pharmaceutically acceptable salts include acid addition salts which may, for example, be formed by reacting the base form drug with a suitable pharmaceutically acceptable acid.

Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

“prolonged period of time” means a continuous period of time of greater than about 1 hour, preferably, greater than about 4 hours, more preferably, greater than about 8 hours, more preferably greater than about 10 hours, more preferably still, greater than about 14 hours, most preferably, greater than about 14 hours and up to about 24 hours.

“push layer” or “push displacement layer” means a formulation which does not contain pharmaceutical agent and which comprises an osmopolymer. Preferably, the push layer comprises an osmopolymer and an osmoagent. The push layer may further optionally contain one or more inactive ingredients, for example disintegrarits, binders, diluents, lubricants, stabilizers, antioxidants, osmotic agents, colorants, plasticizers, coatings and the like.

“releasing structures” means elements of the dosage form capable of discharging, in a controlled and/or predetermined manner, the contents of the reservoirs of which the controlled release dosage form is comprised. Various embodiments of releasing structures are disclosed herein.

“salt forming agent” shall mean acids which may be used in the preparation of pharmaceutically acceptable salts from base forms of drugs. Representative acids which may be used in the preparation of pharmaceutically acceptable salts include, but are not limited to the following: acids: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, a-oxo-glutaric acid, glycolic acid, hipuric acid, hydrobromic acid, hydrochloric acid, hydroiodic acid, isethionic acid, (+)-L-lactic acid, (±)-DL-lactic acid, lactobionic acid, maleic acid, (−)-L-malic acid, malonic acid, mandelic acid, (±)-DL-mandelic acid, methanesulfonic acid, mucic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, tartaric acid, (+)-L-tartaric acid, thiocyanic acid, p-toluenesulfonic acid and undecylenic acid.

“starting materials that are capable of reacting to form a pharmaceutically acceptable salt form of the drug” refers to substances that can react chemically to produce pharmaceutically acceptable salt form of the drug. One example of such starting materials would be a salt forming agent and a micronized base form of a drug. Another example of such starting materials would be a salt forming agent and a liquid base form of a drug.

“subject” or “patient” are used interchangeably herein, and refer to an animal, preferably, a mammal, most preferably, a human, who has been or is the object of treatment, observation or experiment.

“substantially separate” means that there is a physically definable boundary space between the upper gastrointestinal system pharmaceutically acceptable salt form releasing structure and the colonic system base form releasing structure.

“substantially coincident” means that there is no physically definable boundary space between the upper gastrointestinal system pharmaceutically acceptable salt form releasing structure and the colonic system micronized base form releasing structure.

“upper G.I. tract” or “upper gastrointestinal system” means the gastrointestinal system from the mouth to the beginning of the large colon.

“upper gastrointestinal system pharmaceutically acceptable salt form releasing structure” means a releasing structure that operates to release a pharmaceutically acceptable salt form of a drug into the upper gastrointestinal system. These structures can be constructed to operate based on estimates of where the inventive dosage form might be located at a given time following dosing to a patient. For instance, if the typical upper gastrointestinal system transit time for a given dosage form averages around 8 hours following dosing, then the upper gastrointestinal system pharmaceutically acceptable salt form releasing structure can be designed to release the pharmaceutically acceptable salt form within the first 8 hours following dosing. The methods of such design are generally conventionally known.

“zero order rate of release” means a rate of release wherein the amount of drug released as a function of time is substantially constant. More particularly, the rate of release of drug as a function of time shall vary by less than about 30%, preferably, less than about 20%, more preferably, less than about 10%, most preferably, less than about 5%, wherein the measurement is taken over the period of time wherein the cumulative release is between about 25% and about 75%, preferably, between about 25% and about 90%.

### Embodiments

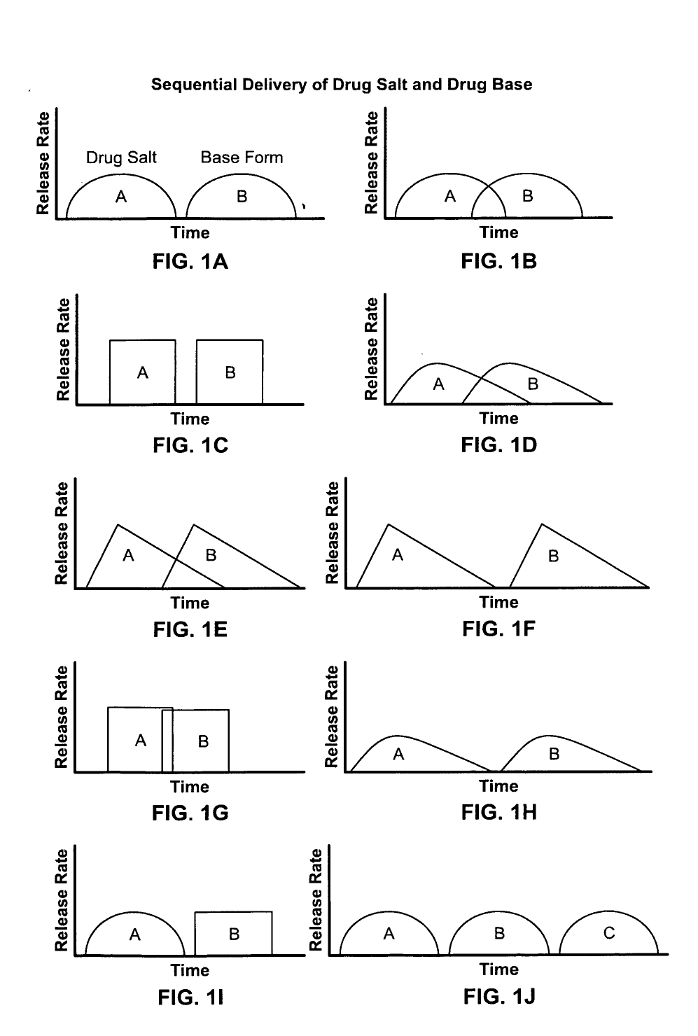

FIGS. 1A through 1J are illustrations of various delivery patterns that can be achieved using this invention. The salt form A would be substantially delivered in the upper gastrointestinal system. Then, the micronized or liquid free base form B would be delivered in the colon. The two patterns can be separate and discrete as illustrated in FIG. 1A, or they can overlap with the end of the first delivered pattern overlapping the beginning of the second delivered pattern as shown in FIG. 1B. Each pattern can be of any desired form and the two waveforms need not be identical. In other words, the dosage forms can be such that the first and second drug forms (salt and base, respectively), deliver different waveforms such as, a square waveform 1C and 1G, an ascending rate form 1D and 1H, a descending rate form 1E and 1F, or any combination of waveforms such as illustrated by FIGS. 1I and 1J.

Examples of useful dosage forms for this embodiment of the invention include, but are not limited to, an osmotic delivery system, a hydrogel matrix containing a plurality of tiny pills, a matrix of drug releasing beads, an immediate release form, or any dosage form capable of providing the desired waveform or delivery profile. Optionally, the salt and base forms of a drug can be physically separated by a membrane. The separation membrane, in addition to compartmentalizing the various drug forms, also serves to prevent possible neutralizing effect that may result at the salt/base interface in the absence of such a membrane. The separation membranes are preferably made of biodegradable polymers which, in the presence of water undergo chemical decomposition and solubilize or decompose to form soluble monomers or polymer units.

In an embodiment, a controlled release dosage form according to the invention comprises a hydrogel matrix dosage form. Such dosage forms preferably comprise a hydrophilic polymer selected from the group consisting of a polysaccharide, hydroxypropyl cellulose, hydroxypropyl methylcellulose, methylcellulose, agar, agarose, natural gum, alkali alginate including sodium alginate, carrageenan, fucoidan, furcellaran, laminaran, hypnea, gum arabic, gum ghafti, gum karaya, gum tragacanth, locust bean gum, pectin, amylopectin, gelatin and a hydrophilic colloid. The hydrogel matrix may optionally comprise a plurality of 4 to 50 drug releasing particles, each drug releasing particle comprising an increasing dose population of from 100 ng ascending in dose such as 0.5 mg, 1 mg, 1.2 mg, 1.4 mg, 1.6 mg, 1.8 mg, etc. The drug releasing particles comprise a release rate controlling wall of 0.0 mm to 10 mm thickness to provide for the timed ascending release of drug. Representative of wall-forming materials include a triglyceryl ester selected from the group consisting of glyceryl tristearate, glyceryl monostearate, glyceryl dipalmitate, glyceryl laureate, glyceryl didecenoate and glyceryl tridecenoate. Other wall forming materials comprise polyvinyl acetate phthalate, methylcellulose phthalate, and microporous vinyl olefins. Procedures for manufacturing drug releasing particles are disclosed in U.S. Pat. Nos. 4,434,153; 4,721,613; 4,853,229; 2,996,431; 3,139,383 and 4,752,470.

Still another controlled release dosage form according to the invention comprises drug releasing beads. Drug releasing beads have been well described in the art and are characterized by a dissolution profile wherein 0 to 20% of the beads undergo dissolution and release the drug in 0 to 2 hours, 20 to 40% undergo dissolution and release the drug in 2 to 4 hours, 40 to 60% exhibit dissolution and release in 4 to 6 hours, 60 to 80% in 6 to 8 hours, and 80 to 100% in 8 to 10 hours. The drug releasing beads comprise a central composition or core comprising a drug and pharmaceutically acceptable composition forming ingredients including a lubricant, antioxidant, and buffer. The beads comprise increasing doses of drug, for example, 1 mg, 2 mg, 5 mg, and 10 mg, increasing to 40 mg. The beads are coated with a release rate controlling polymer that can be selected utilizing the dissolution profile disclosed above. The manufacture of beads is disclosed in Inter. J. of Pharm., by Liu, Vol. 112, pp. 105-116 (1994); Inter. J. of Pharm., by Liu and Yu, Vol. 112, pp. 117-124 (1994); Pharm. Sci., by Remington, 14th Ed. pp. 1626-1628 (1970); J. Pharm. Sci., by Fincher, Vol. 57, pp. 1825-1835 (1968); and U.S. Pat. No. 4,083,949.

One controlled release dosage form according to the invention comprises the osmotic controlled release dosage form. A preferred osmotic controlled release dosage form according to the invention is the multi-layer OROS® drug delivery system (by Alza Corporation, Mountain View, Calif.). The OROS® technology provides tunable controlled release dosage forms that can provide controlled release of drugs and/or pharmaceutically acceptable salts. Various types of osmotic controlled release dosage forms include elementary osmotic pumps, such as those described in U.S. Pat. No. 3,845,770, mini-osmotic pumps such as those described in U.S. Pat. Nos. 3,995,631, 4,034,756 and 4,111,202, and multi-chamber osmotic systems referred to as push-pull, push-melt and push-stick osmotic pumps, such as those described in U.S. Pat. Nos. 4,320,759, 4,327,725, 4,449,983, 4,765,98 and 4,940,465.

A significant advantage to osmotic controlled release dosage forms is that operation is pH-independent and thus continues at the osmotically determined rate throughout an extended time period even as the dosage form transits the gastrointestinal tract and encounters differing microenvironments having significantly different pH values. Controlled release can be provided for times as short as a few hours or for as long as the dosage form resides in the gastrointestinal tract.

Osmotic dosage forms utilize osmotic pressure to generate a driving force for imbibing fluid into a compartment formed, at least in part, by a semi-permeable wall that permits free diffusion of water but not drug or osmagents, if present. In these osmotic controlled release dosage forms, the active agent reservoir(s) is typically formed with an active agent layer, containing a pharmaceutical agent in the form of a solid, liquid or suspension, as the case may be, and an expandable “push” layer of a hydrophilic polymer that will imbibe fluid from the stomach, swell and force the active agent out of the dosage form and into the environment of use. In certain embodiments according to the present invention, the osmotic controlled release dosage form comprises an ion exchange layer.

A review of such osmotic controlled release dosage forms is found in Santus and Baker (1995), “Osmotic drug delivery: a review of the patent literature,” Journal of Controlled Release 35: 1-21. Additionally, the following U.S. patents, are directed to osmotic dosage forms: U.S. Pat. Nos. 3,845,770; 3,916,899; 3,995,631; 4,008,719; 4,111,202; 4,160,020; 4,327,725; 4,519,801; 4,578,075; 4,612,008; 4,681,583; 4,765,989; 4,783,337; 5,019,397; 5,082,668; 5,156,850; 5,912,268; 6,375,978; 6,368,626; 6,342,249; 6,333,050; 6,287,295; 6,283,953; 6,270,787; 6,245,357; and 6,132,420.

In a preferred embodiment, a dosage form of this invention comprises a wall defining a cavity and an exit orifice. Within the cavity and remote from the exit orifice is a push displacement layer, and a drug layer is located within cavity adjacent the exit orifice. An optional flow-promoting layer extends between the drug layer and the inner surface of the wall.

The wall is a semipermeable composition, permeable to the passage of an external fluid, such as water and biological fluids, and substantially impermeable to the passage of active agent, osmagent, osmopolymer and the like. The selectively semipermeable compositions used for forming the wall are essentially nonerodible and are insoluble in biological fluids during the life of the dosage form. The wall need not be semipermeable in its entirety, but at least a portion of the wall can be semipermeable to allow fluid to contact or communicate with the push displacement layer such that the push layer can imbibe fluid and expand during use. The wall preferably comprises a polymer such as a cellulose acylate, cellulose diacylate, cellulose triacylate, including without limitation, cellulose acetate, cellulose diacetate, cellulose triacetate, or mixtures thereof. The wall forming material may also be selected from ethylene vinyl acetate copolymers, polyethylene, copolymers of ethylene, polyolefins including ethylene oxide copolymers such as Engage® (DuPont Dow Elastomers), polyamides, cellulosic materials, polyurethanes, polyether blocked amides copolymers such as PEBAX® (Elf Atochem North America, Inc.), cellulose acetate butyrate, and polyvinyl acetate. Typically, the wall comprises 60 weight percent (wt %) to 100 wt % of the cellulosic wall-forming polymer, or the wall can comprise 0.01 wt % to 50 wt % of polyethylene glycol or ethylene oxide-propylene oxide block copolymers, or 1 wt % to 35 wt % of a cellulose ether selected from the group consisting of hydroxypropylcellulose and hydroxypropylalkylcellulose and 5 wt % to 15 wt % of polyethylene glycol. The total weight percent of all components comprising the wall is equal to 100 wt %.

Representative polymers for forming the wall comprise semipermeable homopolymers, semipermeable copolymers, and the like. Such materials comprise cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semipermeable polymer forming groups, and the like, wherein the organic moieties contain from one to twelve carbon atoms, and preferably from one to eight carbon atoms.

The semipermeable compositions typically include a cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS of 1.8 to 2.3 and an acetyl content of 32 to 39.9%; cellulose diacetate having a DS of 1 to 2 and an acetyl content of 21 to 35%; cellulose triacetate having a DS of 2 to 3 and an acetyl content of 34 to 44.8%; and the like. More specific cellulosic polymers include cellulose propionate having a DS of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a DS of 0.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a DS of 2.6 to 3, such as cellulose trivalerate, cellulose trilamate, cellulose tripalmitate, cellulose trioctanoate and cellulose tripropionate; cellulose diesters having a DS of 2.2 to 2.6, such as cellulose disuccinate, cellulose dipalmitate, cellulose dioctanoate, cellulose dicaprylate, and the like; and mixed cellulose esters, such as cellulose acetate valerate, cellulose acetate succinate, cellulose propionate succinate, cellulose acetate octanoate, cellulose valerate palmitate, cellulose acetate heptanoate, and the like. Semipermeable polymers are known in U.S. Pat. No. 4,077,407, and they can be synthesized by procedures described in Encyclopedia of Polymer Science and Technology, Vol. 3, pp. 325-354, Interscience Publishers Inc., New York, N.Y. (1964).

Additional semipermeable polymers for forming the outer wall comprise cellulose acetaldehyde dimethyl acetate; cellulose acetate ethylcarbamate; cellulose acetate methyl carbamate; cellulose dimethylaminoacetate; semipermeable polyamide; semipermeable polyurethanes; semipermeable sulfonated polystyrenes; cross-linked selectively semipermeable polymers formed by the coprecipitation of an anion and a cation, as disclosed in U.S. Pat. Nos. 3,173,876; 3,276,586; 3,541,005; 3,541,006 and 3,546,142; semipermeable polymers, as disclosed by Loeb, et al. in U.S. Pat. No. 3,133,132; semipermeable polystyrene derivatives; semipermeable poly(sodiumstyrenesulfonate); semi-permeable poly-(vinylbenzyltrimethylammonium chloride); and semipermeable polymers exhibiting a fluid permeability of 10-5 to 10-2 (cc. mil/cm hr. atm), expressed as per atmosphere of hydrostatic or osmotic pressure differences across a semipermeable wall. The polymers are known to the art in U.S. Pat. Nos. 3,845,770; 3,916,899 and 4,160,020; and in Handbook of Common Polymers, Scott and Roff, Eds., CRC Press, Cleveland, Ohio (1971).

The wall may also comprise a flux-regulating agent. The flux regulating agent is a compound added to assist in regulating the fluid permeability or flux through the wall. The flux-regulating agent can be a flux-enhancing agent or a flux-decreasing agent. The agent can be preselected to increase or decrease the liquid flux. Agents that produce a marked increase in permeability to fluid such as water are often essentially hydrophilic, while those that produce a marked decrease to fluids such as water are essentially hydrophobic. The amount of regulator in the wall when incorporated therein generally is from about 0.01% to 20% by weight or more. The flux regulator agents may include polyhydric alcohols, polyalkylene glycols, polyalkylenediols, polyesters of alkylene glycols, and the like. Typical flux enhancers include polyethylene glycol 300, 400, 600, 1500, 4000, 6000 and the like; low molecular weight glycols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glycol dipropionate, glycerol acetate esters, and the like. Presently preferred flux enhancers include the group of difunctional block-copolymer polyoxyalkylene derivatives of propylene glycol known as poloxamers (BASF). Representative flux-decreasing agents include phthalates substituted with an alkyl or alkoxy or with both an alkyl and alkoxy group such as diethyl phthalate, dimethoxyethyl phthalate, dimethyl phthalate, and [di(2-ethylhexyl) phthalate], aryl phthalates such as triphenyl phthalate, and butyl benzyl phthalate; insoluble salts such as calcium sulfate, barium sulfate, calcium phosphate, and the like; insoluble oxides such as titanium oxide; polymers in powder, granule and like form such as polystyrene, polymethylmethacrylate, polycarbonate, and polysulfone; esters such as citric acid esters esterified with long chain alkyl groups; inert and substantially water impermeable fillers; resins compatible with cellulose based wall forming materials, and the like.

Other materials that may be included in the semipermeable wall material for imparting flexibility and elongation properties to the wall, for making the wall less brittle to nonbrittle and to render tear strength. Suitable materials include phthalate plasticizers such as dibenzyl phthalate, dihexyl phthalate, butyl octyl phthalate, straight chain phthalates of six to eleven carbons, di-isononyl phthalate, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

The semi-permeable wall material is dissolved in an appropriate solvent such as acetone or methylene chloride and is then applied to the pressed shape by molding, air spraying, dipping or brushing a solvent-based solution of the wall material onto the shape, as described in U.S. Pat. Nos. 4,892,778 and 4,285,987. Other methods for applying the semi-permeable wall include an air suspension procedure, where the pressed shape is suspended and tumbled in a current of air and wall forming material as described in U.S. Pat. No. 2,799,241, and a pan coating technique.

After application of the semi-permeable wall to the pressed shape, a drying step is generally required and, then, suitable exit(s) for the active agent must be formed through the semi-permeable membrane. The exit orifice cooperates with the drug layer for the uniform release of drug from the dosage form. The exit orifice can be provided during the manufacture of the dosage form or during drug delivery by the dosage form in a fluid environment of use. The dosage form can be constructed with one or more exits in spaced apart relation or one or more surfaces of the dosage form. The exit orifice can be performed by drilling, including mechanical and laser drilling, through the outer coat, the inner coat, or both. Exits and equipment for forming exits are disclosed in U.S. Pat. Nos. 3,845,770; 3,916,899; 4,063,064; 4,088,864, and 4,816,263.

The exit orifice may range in size from a single large orifice containing substantially an entire surface of the dosage form to one or more small orifices selectively located on the surface of the semi-permeable membrane. The exit orifice may be from 10% to 100% of the inner diameter of the compartment formed by wall, preferably from 30% to 100%, and most preferably from 50% to 100%. In addition, in some embodiments, the osmotically controlled dosage form is in the form of an extruded tube open at one or both ends, as described in U.S. Pat. No. 6,491,683 to Dong, et al. In the extruded tube embodiment, it is not necessary to provide an additional exit means. The exit orifice can have any shape, such as round, triangular, square, elliptical and the like for the uniform metered dose release of a drug from the dosage form.

Orifices can also be formed by leaching as disclosed in U.S. Pat. Nos. 4,200,098 and 4,285,987. An exit, or a plurality of exits, can be formed by leaching from outer and/or inner coats one or more of the followng: sorbitol, lactose, fructose, glucose, mannose, galactose, talose, sodium chloride, potassium chloride, sodium citrate, mannitol, erodible poly(glycolic) acid or poly(lactic) acid, gelatinous filament(s); a water-removable poly(vinyl alcohol); inorganic and organic soluble salts, soluble oxides, and leachable polysaccharides.

A flow-promoting layer (also called a subcoat) is optionally in contacting relationship with the inner surface of the semipermeable wall and at least the external surface of the drug layer that is opposite the wall; although the flow-promoting layer may, and preferably will, extend to, surround and contact the external surface of the push displacement layer. The wall typically will surround at least that portion of the external surface of the drug layer that is opposite the internal surface of the wall. The flow-promoting layer may be formed as a coating applied over the compressed core comprising the drug layer and the push layer. The outer semipermeable wall surrounds and encases the inner flow-promoting layer. The flow-promoting layer is preferably formed as a subcoat of at least the surface of the drug layer, and optionally the entire external surface of the compacted drug layer and the push displacement layer. When the semipermeable wall is formed as a coat of the composite formed from the drug layer, the push layer and the flow-promoting layer, contact of the semipermeable wall with the flow-promoting layer may be assured.

The flow-promoting layer facilitates release of drug from the dosage forms of the invention by reducing the frictional forces between the semipermeable wall and the outer surface of the drug layer, thus allowing for more complete delivery of drug from the device. Particularly in the case of active compounds having a high cost, such an improvement presents substantial economic advantages since it is not necessary to load the drug layer with an excess of drug to insure that the minimal amount of drug required will be delivered.

The flow-promoting layer typically may be 0.01 to 5 mm thick, more typically 0.5 to 5 mm thick, and it comprises a member selected from hydrogels, gelatin, low molecular weight polyethylene oxides (e.g., less than 100,000 MW), hydroxyalkylcelluloses (e.g., hydroxyethylcellulose), hydroxypropylcelluloses, hydroxyisopropylcelluoses, hydroxybutylcelluloses and hydroxyphenylcelluloses, and hydroxyalkyl alkylcelluloses (e.g., hydroxypropyl methylcellulose), and mixtures thereof. The hydroxyalkylcelluloses comprise polymers having a 9,500 to 1,250,000 number-average molecular weight. For example, hydroxypropyl celluloses having number average molecular weights of between 80,000 to 850,000 are useful. The flow promoting layer may be prepared from conventional solutions or suspensions of the aforementioned materials in aqueous solvents or inert organic solvents. Preferred materials for the subcoat or flow promoting layer include hydroxypropyl cellulose, hydroxyethyl cellulose, hydroxypropyl methyl cellulose, povidone [poly(vinylpyrrolidone)], polyethylene glycol, and mixtures thereof. More preferred are mixtures of hydroxypropyl cellulose and povidone, prepared in organic solvents, particularly organic polar solvents such as lower alkanols having 1-8 carbon atoms, preferably ethanol, mixtures of hydroxyethyl cellulose and hydroxypropyl methyl cellulose prepared in aqueous solution, and mixtures of hydroxyethyl cellulose and polyethylene glycol prepared in aqueous solution. Most preferably, the flow-promoting layer consists of a mixture of hydroxypropyl cellulose and povidone prepared in ethanol. Conveniently, the weight of the flow-promoting layer applied to the bilayer core may be correlated with the thickness of the flow-promoting layer and residual drug remaining in a dosage form in a release rate assay such as described herein. During manufacturing operations, the thickness of the flow-promoting layer may be controlled by controlling the weight of the subcoat taken up in the coating operation. When the flow-promoting layer is formed as a subcoat, i.e., by coating onto the tableted bilayer composite drug layer and push layer, the subcoat can fill in surface irregularities formed on the bilayer core by the tableting process. The resulting smooth external surface facilitates slippage between the coated bilayer composite and the semipermeable wall during dispensing of the drug, resulting in a lower amount of residual drug composition remaining in the device at the end of the dosing period. When the flow-promoting layer is fabricated of a gel-forming material, contact with water in the environment of use facilitates formation of a gel or gel-like inner coat having a viscosity that may promote and enhance slippage between the semipermeable wall and the drug layer.

The drug layer may additionally comprise a disintegrant, a surfactant, a binding agent, and/or a gelling agent, or mixtures thereof. The binding agent is generally a hydrophilic polymer that contributes to the uniform release rate of active agent and controlled delivery pattern, such as a hydroxyalkylcellulose, a hydroxypropylalkylcellulose, a poly(alkylene)oxide, or a polyvinylpyrrolidone, or mixtures thereof. Representative examples of these hydrophilic polymers are poly(alkylene oxides) of 100,000 to 750,000 number-average molecular weight, including without limitation poly(ethylene oxide), poly(methylene oxide), poly(butylene oxide) and poly(hexylene oxide); poly(carboxymethylcelluloses) of 40,000 to 400,000 number-average molecular weight, represented by poly(alkali carboxymethylcellulose), such as poly(sodium carboxymethylcellulose), poly(potassium carboxymethylcellulose) and poly(lithium carboxymethylcellulose); hydroxyalkylcelluloses of 9,200 to 125,000 number-average molecular weight such as hydroxypropylcellulose, hydroxypropylalkylcelluloses such as hydroxypropylalkylcellulose of 9,200 to 125,000 number-average molecular weight, including without limitation, hydroxypropylethylcellulose, hydroxypropyl methylcellulose, hydroxypropylbutylcellulose and hydroxypropylpentylcellulose; and poly(vinylpyrrolidones) of 7,000 to 75,000 number-average molecular weight. Preferred among those polymers are the poly(ethylene oxide) of 100,000-300,000 number average molecular weight and hydroalkylcelluloses. Carriers that erode in the gastric environment, i.e., bioerodible carriers, are especially preferred.

Surfactants and disintegrants may be utilized in the carrier as well. Disintegrants generally include starches, clays, celluloses, algins and gums and crosslinked starches, celluloses and polymers. Representative disintegrants include corn starch, potato starch, croscarmellose, crospovidone, sodium starch glycolate, Veegum HV, methylcellulose, agar, bentonite, carboxymethylcellulose, alginic acid, guar gum and the like. A preferred disintegrant is croscarmellose sodium.

Exemplary surfactants are those having an HLB value of between about 10-25, such as polyethylene glycol 400 monostearate, polyoxyethylene-4-sorbitan monolaurate, polyoxyethylene-20-sorbitan monooleate, polyoxyethylene-20-sorbitan monopalmitate, polyoxyethylene-20-monolaurate, polyoxyethylene-40-stearate, sodium oleate and the like. Surfactants that are useful generally include ionic surfactants, including anionic, cationic, and zwitterionic surfactants, and nonionic surfactants. Nonionic surfactants are preferred in certain embodiments and include, for example, polyoxyl stearates such as polyoxyl 40 stearate, polyoxyl 50 stearate, polyoxyl 100 stearate, polyoxyl 12 distearate, polyoxyl 32 distearate, and polyoxyl 150 distearate, and other Myrj™ series of surfactants, or mixtures thereof. Yet another class of surfactant useful in forming the dissolved drug are the triblock co-polymers of ethylene oxide/propylene oxide/ethylene oxide, also known as poloxamers, available under the tradenames Pluronic and Poloxamer. In this class of surfactants, the hydrophilic ethylene oxide ends of the surfactant molecule and the hydrophobic midblock of propylene oxide of the surfactant molecule serve to dissolve and suspend the drug. These surfactants are solid at room temperature. Other useful surfactants include sugar ester surfactants, sorbitan fatty acid esters such as sorbitan monolaurate, sorbitan monopalmitate, sorbitan monostearate, sorbitan tristearate, and other Span™ series surfactants, glycerol fatty acid esters such as glycerol monostearate, polyoxyethylene derivatives such as polyoxyethylene ethers of high molecular weight aliphatic alcohols (e.g., Brij 30, 35, 58, 78 and 99) polyoxyethylene stearate (self emulsifying), polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 23 lauryl ether, polyoxyethylene 2 cetyl ether with butylated hydroxyanisole, polyoxyethylene 10 cetyl ether, polyoxyethylene 20 cetyl ether, polyoxyethylene 2 stearyl ether, polyoxyethylene 10 stearyl ether, polyoxyethylene 20 stearyl ether, polyoxyethylene 21 stearyl ether, polyoxyethylene 20 oleyl ether, polyoxyethylene 40 stearate, polyoxyethylene 50 stearate, polyoxyethylene 100 stearate, polyoxyethylene derivatives of fatty acid esters of sorbitan such as polyoxyethylene 4 sorbitan monostearate, polyoxyethylene 20 sorbitan tristearate, and other Tween™ series of surfactants, phospholipids and phospholipid fatty acid derivatives such as lecithins, fatty amine oxides, fatty acid alkanolamides, propylene glycol monoesters and monoglycerides, such as hydrogenated palm oil monoglyceride, hydrogenated soybean oil monoglyceride, hydrogenated palm stearine monoglyceride, hydrogenated vegetable monoglyceride, hydrogenated cottonseed oil monoglyceride, refined palm oil monoglyceride, partially hydrogenated soybean oil monoglyceride, cotton seed oil monoglyceride sunflower oil monoglyceride, sunflower oil monoglyceride, canola oil monoglyceride, succinylated monoglycerides, acetylated monoglyceride, acetylated hydrogenated vegetable oil monoglyceride, acetylated hydrogenated coconut oil monoglyceride, acetylated hydrogenated soybean oil monoglyceride, glycerol monostearate, monoglycerides with hydrogenated soybean oil, monoglycerides with hydrogenated palm oil, succinylated monoglycerides and monoglycerides, monoglycerides and rapeseed oil, monoglycerides and cottonseed oils, monoglycerides with propylene glycol monoester sodium stearoyl lactylate silicon dioxide, diglycerides, triglycerides, polyoxyethylene steroidal esters, Triton-X series of surfactants produced from octylphenol polymerized with ethylene oxide, where the number “100” in the trade name is indirectly related to the number of ethylene oxide units in the structure, (e.g., Triton X-100™ has an average of N=9.5 ethylene oxide units per molecule, with an average molecular weight of 625) and having lower and higher mole adducts present in lesser amounts in commercial products, as well as compounds having a similar structure to Triton X-100™, including Igepal CA-630™ and Nonidet P-40M (NP40TM, N-lauroylsarcosine, Sigma Chemical Co., St. Louis, Mo.), and the like. Any of the above surfactants can also include optional added preservatives such as butylated hydroxyanisole and citric acid. In addition, any hydrocarbon chains in the surfactant molecules can be saturated or unsaturated, hydrogenated or unhydrogenated.

An especially preferred family of surfactants are the poloxamer surfactants, which are a:b:a triblock co-polymers of ethylene oxide:propylene oxide:ethylene oxide. The “a” and “b” represent the average number of monomer units for each block of the polymer chain. These surfactants are commercially available from BASF Corporation of Mount Olive, N.J., in a variety of different molecular weights and with different values of “a” and “b” blocks. For example, Lutrol F127 has a molecular weight range of 9,840 to 14,600 and where “a” is approximately 101 and “b” is approximately 56, Lutrol F87 represents a molecular weight of 6,840 to 8,830 where “a” is 64 and “b” is 37, Lutrol F108 represents an average molecular weight of 12,700 to 17,400 where “a” is 141 and “b” is 44, and Lutrol F68 represents an average molecular weight of 7,680 to 9,510 where “a” has a value of about 80 and “b” has a value of about 27.

Other particularly preferred surfactants are the sugar ester surfactants, which are sugar esters of fatty acids. Such sugar ester surfactants include sugar fatty acid monoesters, sugar fatty acid diesters, triesters, tetraesters, or mixtures thereof, although mono- and di-esters are most preferred. Preferably, the sugar fatty acid monoester comprises a fatty acid having from 6 to 24 carbon atoms, which may be linear or branched, or saturated or unsaturated C6 to C24 fatty acids. The C6 to C24 fatty acids include C6, C7, C8, C9, C10, C11, C12, C13, C14, C15, C16, C17, C18, C19, C20, C21, C22, C23, and C24 in any subrange or combination. These esters are preferably chosen from stearates, behenates, cocoates, arachidonates, palmitates, myristates, laurates, carprates, oleates, laurates and their mixtures.

Preferably, the sugar fatty acid monoester comprises at least one saccharide unit, such as sucrose, maltose, glucose, fructose, mannose, galactose, arabinose, xylose, lactose, sorbitol, trehalose or methylglucose. Disaccharide esters such as sucrose esters are most preferable, and include sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose polyesters, such as sucrose pentaoleate, hexaoleate, heptaoleate or octooleate, and mixed esters, such as sucrose palmitate/stearate.

Particularly preferred examples of these sugar ester surfactants include those sold by the company Croda Inc of Parsippany, N.J. under the names Crodesta F10, F50, F160, and F110 denoting various mono-, di- and mono/di ester mixtures comprising sucrose stearates, manufactured using a method that controls the degree of esterification, such as described in U.S. Pat. No. 3,480,616. These preferred sugar ester surfactants provide the added benefit of tableting ease and nonsmearing granulation.

Use may also be made of those sold by the company Mitsubishi under the name Ryoto Sugar esters, for example under the reference B370 corresponding to sucrose behenate formed of 20% monoester and 80% di-, tri- and polyester. Use may also be made of the sucrose mono- and dipalmitate/stearate sold by the company Goldschmidt under the name “Tegosoft PSE”. Use may also be made of a mixture of these various products. The sugar ester can also be present in admixture with another compound not derived from sugar; and a preferred example includes the mixture of sorbitan stearate and of sucrose cocoate sold under the name “Aratone 2121” by the company ICI. Other sugar esters include, for example, glucose trioleate, galactose di-, tri-, tetra- or pentaoleate, arabinose di-, tri- or tetralinoleate or xylose di-, tri- or tetralinoleate, or mixtures thereof. Other sugar esters of fatty acids include esters of methylglucose include the distearate of methylglucose and of polyglycerol-3 sold by the company Goldschmidt under the name of Tegocare 450. Glucose or maltose monoesters can also be included, such as methyl O-hexadecanoyl-6-D-glucoside and O-hexadecanoyl-6-D-maltose. Certain other sugar ester surfactants include oxyethylenated esters of fatty acid and of sugar include oxyethylenated derivatives such as PEG-20 methylglucose sesquistearate, sold under the name “Glucamate SSE20”, by the company Amerchol.

A resource of surfactants including solid surfactants and their properties is available in McCutcheon's Detergents and Emulsifiers, International Edition 1979. Other sources of information on properties of solid surfactants include BASF Technical Bulletin Pluronic & Tetronic Surfactants 1999 and General Characteristics of Surfactants from ICI Americas Bulletin 0□1 10/80 5M, and Eastman Food Emulsifiers Bulletin ZM-1K October 1993.

One of the characteristics of surfactants tabulated in these references is the HLB value, or hydrophilic lipophilic balance value. This value represents the relative hydroplicility and relative hydrophobicity of a surfactant molecule. Generally, the higher the HLB value, the greater the hydrophilicity of the surfactant while the lower the HLB value, the greater the hydrophobicity. For the Lutrol molecules, for example, the ethylene oxide fraction represents the hydrophilic moiety and the propylene oxide fraction represents the hydrophobic fraction. The HLB values of Lutrol F127, F87, F108, and F68 are respectively 22.0, 24.0, 27.0, and 29.0. The preferred sugar ester surfactants provide HLB values in the range of about 3 to about 15. The most preferred sugar ester surfactant, Crodesta F160 is characterized by having a HLB value of 14.5.

Ionic surfactants include cholic acids and derivatives of cholic acid such as deoxycholic acid, ursodeoxycholic acid, taurocholic acid, taurodeoxycholic acid, taurochenodeoxycholic acid, and salts thereof, and anionic surfactants, the most common example of which is sodium dodecyi (or lauryl)sulfate. Zwitterionic or amphoteric surfactants generally include a carboxylate or phosphate group as the anion and an amino or quaternary ammonium moiety as the cation. These include, for example, various polypeptides, proteins, alkyl betaines, and natural phospholipids such as lecithins and cephalins, alkyl-beta-aminopropionates and 2-alkyl-imidazoline quaternary ammonium salts, as well as the CHAPS series of surfactants (e.g., 3-[3-Cholamidopropyl)dimethylammoniol]-1-propanesulfonate hydrate available from Aldrich), and the like.

Surfactant can be included as one surfactant or as a blend of surfactants. The surfactants are selected such that they have values that promote the dissolution and solubility of the drug. A high HLB surfactant can be blended with a surfactant of low HLB to achieve a net HLB value that is between them, if a particular drug requires the intermediate HLB value. The surfactant is selected depending upon the drug being delivered; such that the appropriate HLB grade is utilized.

The drug layer may be formed as a mixture comprising: (1) a micronized or liquid base form of a drug; (2) either (i) a pharmaceutically acceptable salt form of the drug or (ii) starting materials that are capable of reacting to form a pharmaceutically acceptable salt form of the drug; and/or (3) binding agents and other ingredients. The drug layer can be formed from particles by comminution, typically as a core containing the compound, according to the mode and the manner of the invention. The means for producing particles include granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing the drug and binding agent are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

Generally, liquid base forms of a drug may be converted to a free-flowing powder suitable for tabletting by mixing the liquid drug with a porous, solid carrier. The liquid base form absorbs into the carrier and becomes a free flowing powder. The mixing is typically performed by charging the porous carrier into a twin shell blender, a low shear blender so that the porous carrier is not crushed and the porosity is retained. Then, the liquid base form of a drug is slowly added or sprayed onto the porous carrier with gentle tumbling. This results in the final free-flowing composition that is then compressed into the tablet form. Variations on this process are conventionally known.

The drug layer is typically a dry composition formed by compression of the binding agent and the drug as one layer and the expandable or push layer as the second layer. Compression techniques are known in the art, and are described herein. The push layer pushes the drug layer from the exit orifice as the push layer imbibes fluid from the environment of use, and the drug layer is released into the environment of use.

The push layer is an expandable layer having a push-displacement composition in direct or indirect contacting layered arrangement with the drug layer. The push layer generally comprises a polymer that imbibes an aqueous or biological fluid and swells to push the drug composition through the exit means of the device. Representatives of fluid-imbibing displacement polymers comprise members selected from poly(alkylene oxide) of 1 million to 15 million number-average molecular weight, as represented by poly(ethylene oxide) and poly(alkali carboxymethylcellulose) of 500,000 to 3,500,000 number-average molecular weight, wherein the alkali is sodium, potassium or lithium. Examples of additional polymers for the formulation of the push-displacement composition comprise osmopolymers comprising polymers that form hydrogels, such as Carbopol® acidic carboxypolymer, a polymer of acrylic cross-linked with a polyallyl sucrose, also known as carboxypolymethylene, and carboxyvinyl polymer having a molecular weight of 250,000 to 4,000,000; Cyanamer® polyacrylamides; cross-linked water swellable indenemaleic anhydride polymers; Good-rite® polyacrylic acid having a molecular weight of 80,000 to 200,000; Aqua-Keeps® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No. 4,207,893, issued to Michaels; and in Handbook of Common Polymers, Scott and Roff, Chemical Rubber Co., Cleveland, Ohio.

The osmagent, also known as osmotic solute and osmotically effective agent, which exhibits an osmotic pressure gradient across the outer wall and subcoat, comprises a member selected from the group consisting of sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid raffinose, sucrose, glucose, lactose, sorbitol, inorganic salts, organic salts and carbohydrates.

Exemplary solvents suitable for manufacturing the respective walls, layers, coatings and subcoatings utilized in the dosage forms of the invention comprise aqueous and inert organic solvents that do not adversely harm the materials utilized to fabricate the dosage forms. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

Pan coating may be conveniently used to provide the completed dosage form, except for the exit orifice. In the pan coating system, the subcoat of the wall-forming compositions can be deposited by successive spraying of the respective composition on the bilayered core comprising the drug layer and the push layer accompanied by tumbling in a rotating pan. A pan coater can be used because of its availability at commercial scale. Other techniques can be used for coating the drug core. The coated dosage form can be dried in a forced-air oven, or in a temperature and humidity controlled oven to free the dosage form of solvent. Drying conditions will be conventionally chosen on the basis of available equipment, ambient conditions, solvents, coatings, coating thickness, and the like.

Other coating techniques can also be employed. For example, the semipermeable wall and the subcoat of the dosage form can be formed in one technique using the air-suspension procedure. This procedure consists of suspending and tumbling the bilayer core in a current of air, an inner subcoat composition and an outer semipermeable wall forming composition, until, in either operation, the subcoat and the outer wall coat is applied to the bilayer core. The air-suspension procedure is well suited for independently forming the wall of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, methylene dichloride methanol as a cosolvent. An Aeromatic®air-suspension coater can be used employing a cosolvent.

The dosage form of the invention may be manufactured by standard techniques. For example, the dosage form may be manufactured by the wet granulation technique. In the wet granulation technique, the drug and the ingredients comprising the drug layer are blended using an organic solvent, such as denatured anhydrous ethanol, as the granulation fluid. The ingredients forming the drug layer are individually passed through a preselected screen and then thoroughly blended in a mixer. Next, other ingredients comprising the first layer can be dissolved in a portion of the granulation fluid, such as the solvent described above. Then, the latter prepared wet blend is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate is added to the drug granulation, then put into milling jars and mixed on a jar mill for 10 minutes. The composition is pressed into a layer, for example, in a Manesty® press. The speed of the press may be set at 20 rpm and the maximum load may be set at 2 tons. The first layer may be pressed against the composition forming the second layer and the bilayer tablets may be fed to the Kilian® Dry Coater press and surrounded with the drug-free coat, followed by the exterior wall solvent coating.

In another manufacture the drug and other ingredients comprising the drug layer facing the exit means are blended and pressed into a solid layer. The layer possesses dimensions that correspond to the internal dimensions of the area the layer is to occupy in the dosage form, and it also possesses dimensions corresponding to the push layer for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, the push layer, e.g., a layer of osmopolymer composition, is placed in contact with the layer of drug in a like manner. The layering of the drug layer and the push layer can be fabricated by conventional two-layer press techniques. The two contacted layers are first coated with the flow-promoting subcoat and then an outer semipermeable wall. The air-suspension and air-tumbling procedures comprise in suspending and tumbling the pressed, contacting first and second layers in a current of air containing the delayed-forming composition until the drug and push layers are surrounded by the wall composition.

Another manufacturing process that can be used for providing the compartment-forming composition comprises blending the powdered ingredients in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a tote or V-blender. The granules are then pressed in the manner described above.

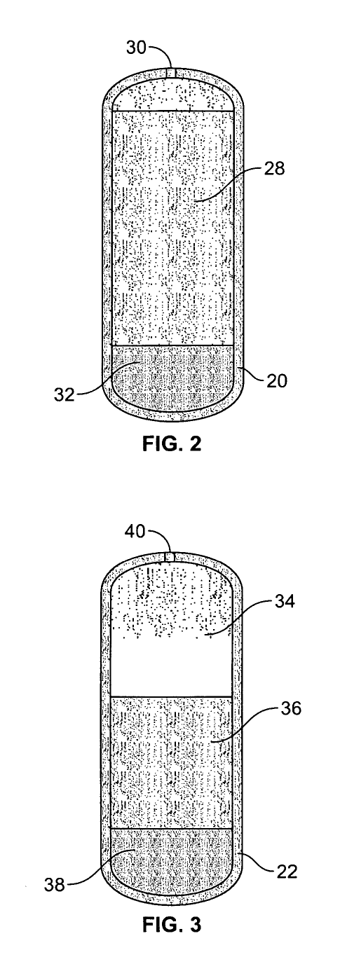

Turning to certain embodiments of the invention, FIG. 2 illustrates an osmotic controlled release dosage form according to the invention. This embodiment comprises an embodiment wherein the upper gastrointestinal system pharmaceutically acceptable salt form releasing structure and the colonic system base form releasing structure are substantially coincident. The salt form is delivered into the upper gastrointestinal system, and the micronized or liquid free base form is delivered into the colon. FIG. 2 illustrates a bilayer core surrounded by a semi-permeable membrane 20. The core layer 28 nearest the delivery port 30 is the drug layer. In FIG. 2, the drug layer comprises drug in micronized or liquid free base form and the same drug in salt form as a homogeneous mixture of each. The drug layer also comprises a polymer that forms a hydrogel in situ when exposed to water. The layer most distant from the delivery port 30 is an osmotic push layer 32. It is formulated with a high molecular weight polymer and an osmotic salt. An example of a push layer composition is a blend of polyoxyethylene 5 million molecular weight and 30% sodium chloride.

When this system is placed in an aqueous environment, the salt form of the drug is pumped preferentially. The preferential pumping takes place while the dosage form is located in the upper gastrointestinal system after dosing. The preferential pumping is due to the higher osmotic driving force of the salt drug compared to the micronized or liquid base form of the drug that has a lower solubility. As the salt form is dispensed from the drug layer, the push layer continuously expands to take up the volume in the drug layer that is evacuated by the soluble drug being dispensed. Once the salt form is dispensed, the remaining lower solubility micronized or liquid base form of the drug is pumped out as a suspension, while the dosage form is located in the colon after dosing. The suspension is pumped from the system by the continued expansion of the push layer. The resulting pattern is similar to those illustrated in FIG. 1.

Another embodiment according to the invention is similar to the dosage form just described except the drug layer is formulated with a homogeneous mixture of the micronized base form of a drug and a salt-forming agent. An example of such a drug base/salt-forming agent is micronized hydrocodone base/tartaric acid. Again, this embodiment comprises an embodiment wherein the upper gastrointestinal system pharmaceutically acceptable salt form releasing structure and the colonic system micronized base form releasing structure are substantially coincident. The salt form is delivered into the upper gastrointestinal system, and the micronized base form is delivered into the colon.

When this system is placed in an aqueous environment, the water imbibed by the system dissolves the tartaric acid and immediately forms in situ the soluble tartrate salt of the drug. The resulting soluble in situ formed salt has greater osmotic activity than the base form and therefore is dispensed preferentially into the upper gastrointestinal system. As the soluble form of the drug is dispensed, the expanding push layer fills the solid volume evacuated by the dissolution of the soluble drug. This process proceeds until the tartaric acid is depleted. At this point, the push layer continues to pump the micronized hydrocodone base as a suspension for a prolonged period of time.

In this embodiment, the duration and extent of salt pumping can be controlled by the amount of salt-forming agent initially present within the drug layer. Increasing the level of tartaric acid, for example, increases the fraction of drug delivered as the tartrate salt and decreases the fraction of drug delivered as the base. Likewise, decreasing the level of tartaric acid decreases the fraction of drug delivered as the salt and increases the fraction delivered as the base.

In this embodiment, the duration and shape of the each drug profile can thus be controlled by the amount of salt forming agent present in the drug layer. The upper gastrointestinal system pharmaceutically acceptable salt form releasing structure releases the salt form of the drug while the dosage form is located in the upper gastrointestinal system following dosing to a patient. The colonic system base form releasing structure then functions to deliver the micronized base form while the dosage form is located in the colon.

FIG. 3 illustrates an embodiment of the invention wherein the upper gastrointestinal system pharmaceutically acceptable salt form releasing structure and the colonic system base form releasing structure are substantially separate. The upper gastrointestinal system pharmaceutically acceptable salt form releasing structure functions to deliver the salt form while the dosage form is located in the upper gastrointestinal system following dosing, and the colonic system base form releasing structure functions to release micronized or liquid base form of the drug while the dosage form is located in the colon following dosing. FIG. 3 illustrates an osmotic system with a tri-layer core surrounded by a semipermeable membrane 22. The first delivered layer 34 is a salt form of a drug. This first delivered layer is optionally formulated with a viscosity-producing agent. An example of a salt form and viscosity agent is a blend of venlafaxine hydrochloride and about 10 weight percent of polyoxyethylene with a molecular weight 200,000 grams per mole. The second drug layer 36 comprises venlafaxine base. This layer is optionally formulated with osmotic agents, such as sodium chloride or sorbitol or with a viscosity-forming agent. The push layer 38 comprises a composition essentially equivalent as that described above.

When this system is placed in an aqueous environment, the incoming water hydrates both drug layers. The layer with venlafaxine HCl is pumped first. This is because it is located adjacent to the exit port 40 and because it has a higher osmotic pressure than the base form of the drug. The drug salt is pumped as a solution. The next drug layer with venlafaxine base is pumped second by the continuously expanding push layer to produce a sequential delivery pattern such as described in Example 1.

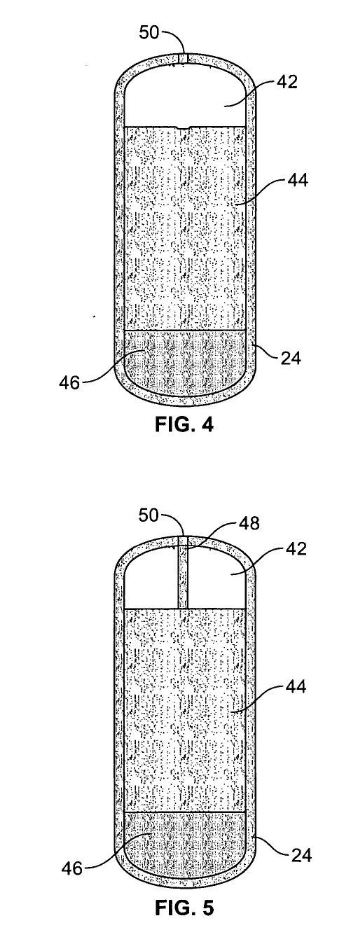

FIG. 4 illustrates a different configuration of the present invention. The core in this embodiment comprises a tri-layer construction. The first layer, located adjacent to the delivery port, comprises a porous cationic exchange resin 42. The resin contains an exchangeable ion such as a hydrogen ion of a carboxyl functional group. The carboxyl functional group is covalently bonded to the backbone of the insoluble resin beads. An example of a pharmaceutical grade resin having such properties is Amberlite IRP64 (available from Rohm and Hass). This resin is of a finely divided size ranging from 25 microns to 150 microns.

Prior to use in the present controlled release dosage form, the resin is preferably granulated with a water-insoluble binder such as ethyl cellulose to provide granules that are larger the size of delivery port of the system. Alternately, the resin can be blended with a polymer having a low glass transition temperature that sinters to a continuous open-cell matrix when compressed. An example of such a polymer is 80/20 polyvinyl acetate/PVP (commercially available from BASF Corporation as Kollidon® SR). The matrix of resin and Kollidon is larger than the size of the delivery port 50 and therefore is retained within the delivery system during operation. The second layer 44 is formulated with a micronized base form of a drug homogeneously mixed with a non-drug salt. An example of a non-drug salt for this embodiment is sodium chloride or potassium chloride. Optionally, the second layer 44 can be formulated to contain a hydrogel-forming polymer such as low molecular weight polyoxyethylene. The third layer 46, located most distant from the delivery port 50, is a push layer composition such as described above.

When placed in an aqueous environment, the non-drug salt dissolves and ionizes. The resulting sodium ion exchanges for the hydrogen ion of the carboxyl group covalently bonded to the immobile resin. The exchange is driven by preferential affinity of carboxyl group for sodium over hydrogen. The relative affinities of certain cations to cationic exchange resins useful in the present invention is as follows:
Ba⁺²>Ca⁺²>Zn⁺²>Mg⁺²>Ag⁺¹>K⁺¹>NH₄⁺¹>Na⁺¹>H⁺¹.

The resulting mobile hydrogen ion next forms hydrochloric acid with the mobile chloride anion. The resulting HCl then immediately reacts with the micronized base form of the drug to form the corresponding HCl salt form of the drug. This soluble form is preferentially pumped through the porous ion exchange membrane layer since it is more soluble than the base form of the drug and therefore generates higher osmotic activity. The cation exchange process continues until hydrogen content of the ion exchange layer is substantially depleted. Then, the remaining unconverted micronized base form of the drug is dispensed as a suspension. The resulting delivery pattern can be of many forms such as depicted in FIG. 1.

This ion exchange embodiment provides the means to deliver HCl salts of drugs. These are by far the most common salt forms that are commercially available. Examples include tramadol HCl, bupropion HCl, ciprofloxacin HCl, metformin HCl, fexofenadine HCl, and ondansetron HCl.

The series of in situ ion exchange reactions illustrating the HCl drug salt formation is summarized as follows:

### Conversion of Insoluble Drug Base to Soluble Drug Salt by Sequential Delivery System

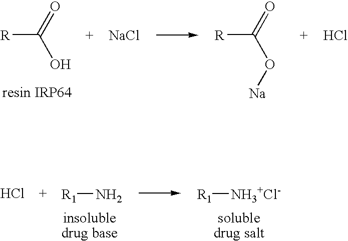

In a different embodiment, a different ion exchange resin may be used in a dosage form similar to the one described above. Amberlite IRP69 is a pharmaceutical grade cationic exchange resin based on sulfonic acid functionality (commercially available from Rohm & Haas Company). This is a strongly acidic exchange resin that exchanges more independently of pH than IRP64. In its commercial form, IRP 69 is a sodium salt. At the present time, a free acid form of this resin is not commercially available. For the purposes of this invention, the acidic exchange resin is first converted to the free acid form by treatment with aqueous hydrochloric acid. The series of reaction steps is as follows:

### Conversion of Cationic Resin from Salt to Free Acid form Prior to use in Delivery System

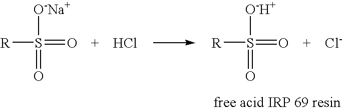

### Conversion of Insoluble Drug Base to Soluble Drug Salt by Sequential Delivery System

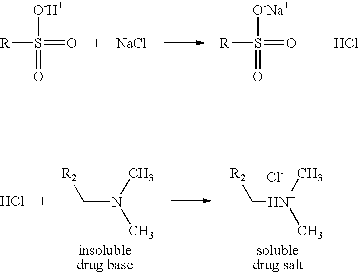

In yet another embodiment of the present invention, an ion exchange osmotic controlled release dosage form is as shown in FIG. 5. The construction of this dosage form is essentially equivalent to the dosage form in FIG. 4 except that in this dosage form, a channel 48 is formed within the ion-exchange layer. The channel provides a continuous pathway through the ion exchange layer and connects the drug layer to the exit orifice as illustrated in FIG. 5.

In operation, the ion exchange process takes place within the channel as well as within the pores of the ion exchange layer, with delivery of the salt form of the drug taking place while the inventive dosage form is located in the upper gastrointestinal system. After the hydrogen ions from the resin are depleted, the remaining unconverted base form of the drug is extruded through the channel while the dosage form is located in the colon. This produces the sequential delivery pattern as illustrated in FIGS. 1A-J.

In another embodiment, a dosage form similar to the system in FIG. 4 is provided, except that at least some of the sodium chloride is formulated within the ion exchange layer. In operation, the imbibed water dissolves the NaCl, resulting in water filled-pores within the ion exchange layer. The open pores promote passage of the drug through the ion exchange layer. The rate of ion exchange would be greater since the NaCl is initially concentrated within the ion exchange layer. This more readily produces the hydrochloric acid available to convert the drug base to the drug salt. The net effect is to shorten onset of drug salt delivery.

In yet another embodiment of the present invention, controlled release dosage forms may be prepared comprising a rate control membrane that is formulated with enteric properties. In operation, the dosage form pumps little if any drug in the stomach. When emptied from the stomach the delivery system begins pumping in a defined portion of the upper gastrointestinal system, namely the small intestine. The dosage form therefore operates in the gastrointestinal tract where pH conditions are relatively mild during the functional lifetime of the dosage form. Also, the enteric coating may increase the likelihood that the base form of the drug is delivered in the lower gastrointestinal tract in certain instances. This is because the osmotic controlled release dosage form is designed to pump for a fixed duration of time of about 12-16 hours but gastric emptying time can vary from less than an hour to 4 hours or more. The enteric membrane reduces the effect of variation in gastric emptying time since the osmotic controlled release dosage form starts pumping once emptied from the stomach.

The following examples are illustrative of the dosage forms, of the present invention and they should not be considered as limiting the scope of this invention in any way, as these examples and other equivalents thereof will become apparent to those versed in the art in the light of the present disclosure and the accompanying claims.

### EXAMPLES

### Example 1

### Ranitidine® Drug Forms

Ranitidine® is indicated for the treatment of gastric and duodenal ulcers. It is typically prescribed as two 150 mg tablets administered twice a day or one 300 mg tablet administered once daily. Therapy typically involves a long dosing regimen of about four weeks or more. Despite this protracted dosing regimen, many patients continue to experience the discomfort of the condition. It is estimated that about 20 to 30% of the patient population remain uncured as not cured even after weeks of therapy. A dosage form that can provide improved therapy by reducing the duration of the dosing regimen and by increasing the fraction of patient population that can be effectively treated with this drug is identified as an unmet medical need.

A sequential osmotic dosage form is developed that provides a first pattern of release comprising the salt form and a second pattern of release comprising the base form of the drug. The resulting delivery system provides an oral dosage form that delivers the salt form in the upper gastrointestinal tract and then delivers the base form in the colon to improve therapy for patients in need.

The dosage form of the present invention comprises a three layer osmotic tablet coated with a semi-permeable, rate-controlling membrane. The dosage form is formulated such that all of the drug prior to dosing is present in the base form of the drug. After dosing, a portion of the drug is converted to a salt form by an ion exchange-based mechanism. The dosage form is designed such that this salt form of the drug is delivered first, in the upper gastrointestinal system, then the base form is delivered to the colon.

The dosage form of this embodiment is fabricated according to the following procedures and compositions. First, a batch of ion exchange resin with strongly acidic functionality is prepared. This is accomplished by converting of a pharmaceutical grade resin copolymer of styrene and divinyl benzene having sodium sulfonate functionality to the sulfonic acid functionality. The resin is commercially available from the Rohm and Haas Company, Philadelphia, Pa., as Amberlite® IRP69. About 1 kilogram of the resin is treated with 1 normal hydrochloric acid until the sodium ions are substantially exchanged with protons according to the following equation: 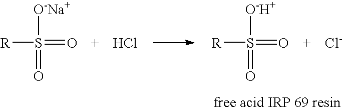
where R represents the styrene divinyl benzene backbone of the resin.

The converted resin is then rinsed with de-ionized water and dried in a forced air oven at 35° C. overnight to remove residual moisture. This forms the protonated cation exchange resin that is incorporated as a functional element of the dosage form of the present invention.

To make this dosage form, about 809 grams of the dried protonated exchange resin and 131.0 grams of sodium chloride powder are passed through a 60-mesh sizing sieve and are transferred to a planetary bowl mixer. Next, 50 grams of cellulose acetate is dissolved in 100 ml of anhydrous acetone with stirring. The cellulose acetate has an average molecular weight of 30,000 and is commercially available from Eastman Chemical as Type CA-398-3. While mixing the powders in the bowl, the polymer solution is slowly added to the powders until a uniform damp mass is formed. The resulting damp mass is then passed through a 20-mesh sieve, forming elongated granules. The resulting elongated granules are tray dried in forced air at 35° C. two days to remove residual acetone. The elongated granules are then passed again through a 20-mesh sieve. The resulting free-flowing granules are transferred to a twin shell mixer. 10.0 grams of stearic acid are sized through a 60-mesh sieve and tumble blended into the granules for 1 minute. This forms tablet layer composition 1.

Tablet layer composition 2 is prepared by sizing through a 40-mesh sieve 840 grams of solid micronized ranitidine base previously micronized in an air jet mill using standard processing procedures according to the manufacturer's instructions, 100 grams of polyoxyethylene having a molecular weight of 200,00 grams per mole (available as Polyox N80), and 50.0 grams of vinylpyrrolidone vinyl acetate copolymer. The copolymer is available as Kollidon VA 64 from BASF Corporation. The sized powders are transferred to a planetary bowl mixer and stirred to a homogeneous blend. 100 ml of anhydrous ethanol SDA 3A (Handbook of Chemistry, Norbert Lange (1941)) is then slowly added while stirring the powders to form a uniform damp mass. The mass is sized through a 20-mesh sieve to form granulate. The granulate is oven dried at 35° C. overnight. The resulting dried granulate is passed through a 20-mesh sieve and is then transferred to a twin shell mixer. Then, about 10 grams of stearic acid is passed through an 80-mesh sieve and tumble mixed into the granules for 1 minute. This forms tablet layer composition 2.

A third granulation is prepared according to the following compositions and procedures. 737.0 grams of polyoxyethylene, 200 grams of sodium chloride, and 50.0 grams of polyvinyl pyrrolidone, are passed through a 40-mesh sieve and transferred to a planetary mixer. 10.0 grams of ferric oxide red and 0.5 gram of butylated hydroxytoluene are passed through a 60-mesh sieve and added to the powders and mixed into the blend. The polyethylene oxide has a molecular weight of 7 million grams per mole and is commercially available as Polyox 303. The polyvinyl pyrrolidone has a molecular weight of approximately 10,000 grams per mole and is commercially available from BASF Corporation, Mount Olive, N.J., as Kollidon 30. 350 ml of anhydrous ethanol are then slowly added to the powders with mixing until a uniform damp mass is produced. The resulting damp mass is then passed through a 20-mesh sieve, forming elongated extrusions. The resulting damp extrusions are air dried overnight and are then passed again through a 20-mesh sieve. The resulting free-flowing granules are transferred to a twin-shell mixer where 2.5 grams of stearic acid, previously sized 60 mesh, are tumble mixed into the granules. This forms the push layer granulation.

Tri-layer tablets are next compressed manually with a Carver bench top press fitted with 17/64-inch diameter tablet punch tooling and dies. First, 130 mg tablet layer composition 1 is filled into the die cavity and lightly tamped. Then, 320 mg of tablet layer composition 2 is filled into the cavity and slightly tamped. Finally, 150 mg of the push layer granulation is filled into the die cavity and compressed with the upper punch using a force of 2500 pounds. Tablet layer composition 2 comprises 268.8 mg of ranitidine base. This weight of base is equivalent on a molar bases to the weight of a 300 mg dose of ranitidine hydrochloride salt. This forms the tri-layer tablet of the present invention.

Preferably, a barrier layer is interposed between the first and second drug layers to prevent possible adverse effects at the salt/base interface such as any neutralizing effects. It is however contemplated that for certain dosage forms, it may be possible or indeed desirable to take advantage of this feature of the invention in order to generate a desired profile.

A coating solution is prepared by dissolving 90 grams of A:B:A tri-block copolymer in 5,700 grams of acetone with warming and stirring. The tri-block copolymer is an ethylene oxide:propylene oxide ethylene:oxide tri-block copolymer having an average molecular weight ranging from 7,680 to 9510. The number of ethylene oxide monomer units per block is approximately 80 and the number of propylene oxide monomer units per block is approximately 27. Then, 210 grams of cellulose acetate is dissolved into he blend with stirring. The cellulose acetate has an average acetyl content of 39.8 weight percent and an average molecular weight of 35,000.

The batch of tri-layer tablets is loaded into a pharmaceutical pan coater. The coating solution is then sprayed onto the bed of tablets as they tumble within the pan in a current of warm drying air until a uniform coating thickness of 5 mils is deposited onto each tablet. A delivery orifice or port having a nominal diameter of 1 mm is drilled on the end of the tablet nearest the ion exchange layer using a mechanical drill. The orifice is drilled through the external rate controlling membrane and through the ion exchange layer 1 to a depth such that it reaches tablet layer 2 composition. The resulting delivery channel therefore connects drug layer composition 2 with the outside environment of the dosage form. The resulting batch of drilled systems is then dried in a forced air oven at 40° C. for 3 days to remove residual coating solvent. This completes fabrication of the sequential delivery system.

When administered orally to a patient in need of anti-ulcer therapy, water from the gastrointestinal tract is imbibed by osmosis across the rate controlling membrane into the three layers of the core. The push layer hydrates and begins to gel and swell and the drug layer composition 2 hydrates and gels. Water simultaneously activates ion exchange mechanism in layer 1 by locally dissolving the sodium chloride present within the ion exchange layer. The resulting mobile sodium ion from the sodium chloride then is available to exchange with the sulfonate proton of the resin to produce aqueous hydrochloric acid according to the following equation:

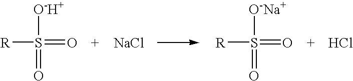

The resulting free hydrochloric acid then associates with the dimethylamino functionality of ranitidine base molecule. This reaction within the dosage form converts the base form of the molecule to the salt form according the following equation:

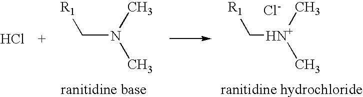

Ranitidine HCl is freely soluble in water with a solubility value at 37° C. of 667 mg per milliliter. The osmotic pressure of the drug is also very high at 95 atmospheres. Therefore, the osmotic driving force for the salt form is very high. The osmotic driving force for ranitidine base is lower. Therefore, the hydrochloride salt of the drug is delivered preferentially at the outset of the release pattern. The HCl salt form of the drug continues to be dispensed from the delivery system as it is continuously formed in the ion exchange layer for several hours until the sodium chloride is depleted in the reaction.

Sufficient sodium chloride is formulated within ion exchange layer to convert approximately one third of the ranitidine base molecules to ranitidine hydrochloride salt molecules. As the conversion is completed, the push layer continues to expand against the hydrated and gelled ranitidine base composition layer 2. Drug layer composition 2 is therefore extruded as a hydrogel paste with suspended particles of ranitidine base carried through the delivery channel over a prolonged period of time.

These mechanisms yield a dosage form providing a sequential delivery pattern of ranitidine hydrochloride dispensed in the upper gastrointestinal tract and then a delivery pattern of ranitidine base dispensed in the lower tract to the patient in need of improved therapy.

### Example 2

### Tizanide® Drug Forms

Tizanidine is a centrally acting muscle relaxant prescribed for symptomatic relief of spasticity associated with multiple sclerosis or spinal chord injury or disease. It is a short acting medication that must be administered three to four times per day to maintain the therapeutic effect. Common side effects are significant and include dry mouth, somnolence, asthenia, or dizziness. These side effects appear to be dose-related as they are less prevalent at lower doses. Therefore, there is a substantial unmet medical need for an oral dosage form of the drug that can be administered once or twice daily with reduced incidence of side effects.

The HCl salt form of the drug has a solubility value in water in the range of 1-10 mg/ml. The salt form is absorbed as a solution in the upper gastrointestinal tract when administered as an immediate release dosage form. The drug solubility is reduced, however, as the pH increases. This pH dependent solubility causes the HCl salt to precipitate in intestinal fluids to particles with sizes that are uncontrolled and are therefore associated with poorly controlled and variable absorption in the colon.

The dosage form of the present invention is designed to first deliver a fraction of the dose in the soluble salt form within the upper gastrointestinal system where the drug is well absorbed as a solution. The dosage form then delivers a second fraction of dose as insoluble base form delivered in a finely divided micronized particle size form within the lower gastrointestinal tract where micronized form is better absorbed and more uniformly absorbed than the precipitated forms having a wider distribution of particle sizes.

The sequential delivery system of this embodiment is fabricated according to the following procedures and compositions. About 163.4 grams of tizanidine hydrochloride, 796.6 grams of Polyox N80, and 30.0 grams of polyvinyl pyrrolidone are sized through a 40-mesh sieve. The polyvinyl pyrrolidone has a molecular weight of approximately 360,000 and is available commercially available from BASF, as Kollidon® 90 F (?).

The sized powders are transferred to a planetary mixer and stirred. While stirring the powders, 200 ml of anhydrous ethanol specially denatured alcohol formula (SDA) 3A is slowly added until a uniform damp mass is produced. The resulting damp mass is extruded through a 20-mesh sieve to form elongated extrusions. The resulting extrusions are dried in a forced air oven at 40° C. overnight to remove the ethanol and are then passed again through a 20-mesh sieve, forming free flowing granules. The resulting granules are then transferred to a twin shell mixer. Then 10 grams of tablet lubricant, stearic acid, are passed through an 80-mesh sieve and added to the granules. To complete the first drug layer granulation, the composition is then tumble mixed for two minutes.

A second drug layer composition is fabricated as follows. Tizanidine base is first micronized in an air jet mill according to the manufacturers instructions to a nominal particle size of 3 to 5 microns. 145.5 grams of the resulting micronized tizanidine base, 814.5 grams of Polyox N80, and 30.0 grams of Kollidon 90 F are then passed through a 40-mesh sizing screen and mixed in a bowl mixer with a planetary mixing blade. While mixing, 250 ml of anhydrous ethanol are slowly added to form a uniform damp mass. The damp mass is then passed through a 20-mesh sieve, dried overnight in a forced air oven at 40° C., and passed again through a 20-mesh sieve. To complete the second drug layer granulation, the resulting granules are lubricated with 10.0 grams of minus 80-mesh stearic acid in a twin shell mixer.

Trilayer tablets are made using 3/16-inch diameter punch and die tooling. First, 60 mg of the push layer composition described in Example 1 are filled into the die cavity and lightly compacted. Then, 110 mg of drug layer 2 granulation containing micronized drug base are added to the cavity and lightly compacted. Finally, 70 mg of first drug layer granulation are fed to the cavity and compressed with a force of 1000 pounds. This produces a trilayer tablet that contains a total unit dose equivalent to 26 mg tizanidine base. The first layer comprises 11.44 mg of the hydrochloric acid salt form of the drug that is equivalent to 10.0 mg as the base form; while the second layer comprises 16.0 mg of tizanidine base. A batch of the tri-layer tablets is then compressed.

In a final coating step, a coating solution is prepared by dissolving 60 grams of A:B:A tri-block copolymer in 5,700 grams of acetone with warming and stirring. The tri-block copolymer is an ethylene oxide:propylene oxide ethylene:oxide tri-block copolymer having an average molecular weight ranging from 7,680 to 9510. The number of ethylene oxide monomer units per block is approximately 80 and the number of propylene oxide monomer units per block is approximately 27. Then, 240 grams of cellulose acetate is dissolved into the blend with stirring. The cellulose acetate has an average acetyl content of 39.8 weight percent and an average molecular weight of 35,000.

The batch of tri-layer tablets is loaded into a pharmaceutical pan coater. The coating solution is then sprayed onto the bed of tablets as they tumble within the pan in a current of warm drying air until a uniform coating thickness of 7 mils is deposited onto each tablet. The coating serves as both a semipermeable rate controlling membrane and a rigid housing defining the dimensions of the dosage form while in operation. A delivery port having a nominal diameter of 1 mm is drilled on the drug layer end of the tablet using a laser drill. To complete fabrication of the sequential delivery system, the resulting batch of drilled systems is dried in a forced air oven at 40° C. for 3 days to remove residual coating solvent. This completes fabrication of the sequential delivery system.

When administered to a patient in need of treatment, the osmotic controlled release dosage form imbibes water from the gastrointestinal tract by osmosis. The incoming water hydrates the three layers of the tablet. As each dosage form layer hydrates, a hydrogel of polyoxyethylene is formed within each layer. As the push layer expands within the fixed volume defined by the rigid rate controlling membrane, the delivery system dispenses the drug through the delivery orifice in a sequential delivery pattern. The drug salt is first delivered then the micronized drug base is delivered. The soluble salt form is well absorbed in the upper gastrointestinal tract while the finely divided insoluble form is well absorbed in the colon to provide a dosage form that is administered less frequently with fewer side effects.

### Example 3

### Propoxyphene® Drug Forms

Propoxyphene is a narcotic analgesic that is prescribed for the relief of mild to moderate pain. The drug is short acting and therefore must be administered every four hours to maintain therapeutic plasma levels to achieve and maintain relief of pain. This dosing regimen is obtrusive to normal daily activity schedules and prevents patients in need from achieving an uninterrupted and restful night sleep. The need for dosage form that can be administered once or twice daily is identified to meet this unmet medical need.

The drug is commercially available in a hydrochloride form and a base form. The hydrochloride form is freely soluble in water and when administered orally is absorbed quickly from the gastrointestinal tract. The base form is only slightly soluble in water and is absorbed slowly. The shortcoming of the individual drug form can be overcome by a dosage form that delivers the soluble salt form of the drug first followed by the lowly soluble form is prepared. The soluble form of the drug provides rapid onset of pain relief while the low solubility drug form delivered continuously over prolonged periods of time provides extended duration of pain relief. The dosage form of the present invention comprises an erodible bilayer tablet for sequential delivery of propoxyphene hydrochloride and propoxyphene base.

The dosage form is fabricated according to the following compositions and procedures. First, 216.7 grams of propoxyphene hydrochloride, 385.0 grams of lactose, and 385.0 grams of microcrystalline cellulose are passed through a sizing screen having 40 wires per inch. The microcrystalline cellulose is supplied as Avicel PH-101 (available from FMC Corporation, Philadelphia, Pa.) The sized powders are transferred to a twin shell blender and tumble mixed for 15 minutes. 3.3 grams of red ferric oxide, and 10.0 grams of magnesium stearate are passed through an 60-mesh sieve and added to the mixed powders. The blend is tumble mixed for two minutes to provide the soluble drug layer composition.

A slow eroding layer composition is prepared as follows. First, a batch of propoxyphene base is micronized in an air jet mill to a nominal particle size of 3-6 microns according to the manufactuers instructions. 500.0 grams of the micronized propoxyphene base are added to a planetary bowl mixer. Then, 440.0 grams of polyoxyethylene and 50.0 grams of hydroxypropyl methyl cellulose are sized through a 40-mesh sieve and added to the bowl. The polyoxyethylene has a molecular weight of approximately 7 million and is available commercially as Polyox Coagulant from the Dow Chemical Company, Midland, Mich. The hydroxypropyl methyl cellulose has a molecular weight of approximately 11,300 and is available from the same vendor as Methocel E5 Premium. The resulting powders are mixed. Then, 300 ml of anhydrous ethyl alcohol SDA 3A anhydrous are slowly added to the mixed powders to form uniform damp mass. The resulting mass is passed through a 16-mesh sizing screen producing elongated granules. The resulting elongated granules are tray dried in a forced air oven at 45° C. for 24 hours to remove residual granulation solvent. The dried mass is then passed again through the 16-mesh sizing screen, producing free flowing granules. The granules are transferred to a twin shell blender. About 10 grams of magnesium stearate are sized through an 80-mesh sieve and added to the granules. The composition is then tumble mixed for about 1 minute to produce the insoluble drug layer composition.

Portions of the insoluble layer composition weighing 600 mg are filled into 7/10-inch long oval punch and die tablet tooling and lightly compressed to partially consolidate the granules. Then, 300 mg of the soluble layer composition are added to the die and the granules and the granules are compacted with a force of 2 tons to form a bilayer tablet. A batch of these bilayer tablets is compressed.

The resulting batch of tablets is transferred to a pharmaceutical pan coater. A taste mask coating solution is prepared by dissolving with stirring 40 grams of hydroxypropyl methyl cellulose and 10 grams of polyethylene glycol in 950 ml of de-ionized water. The hydroxypropyl methyl cellulose has a molecular weight of approximately 11,900 and is commercially available as Pharmacoat 606 from Shin-Etsu Chemical Company, Tokyo, Japan. The polyethylene glycol is commercially available from Dow Chemical as Carbowax 8,000. The resulting coating solution is spray coated onto the bilayer tablets in the pan coating using a stream of warm dry air until approximately 27 mg of coating is applied to each bilayer tablet. The resulting dosage forms are tumble dried in the pan for 30 minutes to remove residual moisture to complete the fabrication of the dosage form. The soluble drug layer composition comprises a dose of 65 mg of propoxyphene hydrochloride while the slow layer comprises a dose of 300 mg of propoxyphene base.

The resulting dosage form is administered orally to a patient in need of prolonged relief of pain. When exposed to the fluids of the gastrointestinal tract, the taste mask layer quickly dissolves and exposes the bi-layer tablet to the aqueous environment. Water is then immediately wicked into the fast layer by the microcrystalline cellulose, causing this layer to rapidly disintegrate and to rapidly release the soluble propoxyphene hydrochloride. This immediate release fraction of the drug dose provides rapid absorption of the drug to provide rapid relief of pain. The high molecular weight polymer of the remaining low solubility drug layer imbibes water slowly, causing the lowly soluble, finely divided propoxyphene base to be slowly released over a period of many hours to maintain prolonged relief of pain for many hours.

### Example 4

### Prochlorperazine® Drug Forms

Prochlorperazine is a medication indicated for a variety of conditions including treatment of nausea and vomiting. The drug is administered orally three or four times a day. Patients experiencing nausea and vomiting often are unable or unwilling to swallow several dosage forms per day. To meet this need, oral dosage forms that can be administered fewer times per day or that can be administered by non-oral routes have been developed. Suppository dosage forms for rectal delivery, for example, are commercially available. This route of administration, however, is often unacceptable to patients. Likewise, parenteral forms of the drug are also available. This route of administration is associated with the pain of needle pricks that are unpleasant, particularly if repeatedly administered every three to four hours. Oral dosage forms have also been developed to meet this need. Current commercially available dosage forms, however, require twice daily administration, and are unacceptable to patients in need of relief from nausea, especially severe nausea. Patients prefer to swallow doses as infrequently as possible, preferably only once daily. No oral product for once daily dosing of prochlorperazine to relieve patients of the need is commercially available.

The drug molecule is commercially available in several salt forms and the base form each of which have different physical properties. The edisilate salt of prochlorperazine, for example, is a hydrophilic, crystalline powder that has a solubility in water of about 500 mg per millilter. The base form is a hydrophobic, viscous oil that has a much lower aqueous solubility that is in the range of 0.1 to 1 mg per milliliter. To demonstrate another aspect of the present invention, an oral osmotic delivery system is fabricated that sequentially delivers the edisilate salt form followed by the oil base form of prochlorperazine for once daily treatment to patients in need of anti-emetic therapy.

The anti-emetic dosage form of the present invention comprises an osmotic tablet and an osmotic rate-controlling coating to regulate the release rate of drug. The tablet is fabricated with three compressed layers arranged in series. The tri-layer tablet is fabricated according to the following procedures and compositions. First, a drug composition is prepared by passing 188.6 grams of prochlorperazine edisilate, 751.4 grams of polyoxyethylene, and 25.0 grams of polyvinyl pyrrolidone are passed through a 40-mesh sizing screen. The polyoxyethylene has a molecular weight of approximately 100,000 and is available commercially from Dow Chemical as Polyox N10. The polyvinyl pyrrolidone has a molecular weight of approximately 10,000 and is available commercially from BASF Corp as Kollidon 30. 25.0 grams of the same polyvinyl pyrrolidone is then dissolved with stirring in 975 ml of de-ionized water. The powders are charged into a Glatt fluid bed granulator and fluidized in a current of warm air. The polyvinyl pyrrolidone solution is sprayed onto the fluidized powders to form granules. The resulting granules are sized through a 16-mesh sieve, and are then transferred to a twin shell mixer. About 10 grams of minus 60 mesh stearic acid is tumble mixed into the granulesto produce the composition of a first drug layer.

A second drug layer composition is prepared by charging 500 grams of porous magnesium aluminometasilicate particles and 250 grams of liquid prochlorperazine base into a twin shell blender. The resulting components are tumble blended for 30 minutes to absorb uniformly the liquid drug into the porous carrier. The porous carrier is available commercially from Fuji Chemical Company, Toyama, Japan as Neusilin US2. About 190 grams of Polyox N10 and 25.0 grams of Kollidon 30 are sized through a 40-mesh sieve and blended into the mixture for 5 minutes. About 25 grams of Kollidon 30 are dissolved with stirring in 575 ml of de-ionized water. The blended powders are charged into a Glatt fluid bed granulator and fluidized in a current of warm air while spraying on the polyvinyl pyrrolidone solution. This process forms granules that after the solution is sprayed are sized through a 16-mesh sieve. The sized granules are transferred to a twin shell mixer and blended for 2 minutes with about 10 grams of minus 60 mesh stearic acid. This process and composition provides free-flowing granules for the second drug layer composition which is the base form of the drug in an oil medium.

Push layer composition is provided by first sizing through a 40-mesh sieve 643.0 grams of polyoxyethylene, 292.0 grams of sodium chloride powder, and 50.0 grams of hydroxypropyl methyl cellulose. The polyoxyethylene has a molecular weight of approximately 5 million and is available commercially from Dow Chemical as Polyox Coagulant. The hydroxypropyl methycellulose has a molecular weight of approximately 11,300 is available from Dow Chemical as Methocel E5. The sized powders are charged into a planetary bowl mixer. About 10 grams of ferric oxide red is sized through a 60-mesh sieve over the powders.

The powders are mixed for several minutes until a uniform color blend is achieved. Then, while mixing the powders, 230 ml of anhydrous ethanol formula SDA 3A is added slowly to form a uniform damp mass. The resulting damp mass is passed though a 20 mesh sizing screen forming extrusions. The extrusions are tray dried in forced air at 45° C. for 40 hours to remove residual ethanol. The dried granules are then passed through a 20-mesh sizing screen and transferred to a twin-shell blender. Finally, 5.0 grams of magnesium stearate are sized through an 80-mesh sieve over the granules and tumble mixed into the granules for two minutes. This procedure and components forms the push layer composition.

Tri-layer tablets of the three layer compositions are compressed manually on a Carver press fitted with 3/16 inch diameter tablet punch tooling and dies. First, 80 mg of the push layer composition is filled into the die cavity and lightly tamped. Next, 80 mg of drug layer composition 2 is filled into the cavity and lightly compacted. Finally, 80 mg of drug layer composition 1 is filled into the die cavity. The layered compositions are then compressed under a final force of 1200 pounds to form the final tri-layer tablet. Drug layer composition 1 in the tablet comprises 15.09 mg of prochlorperazine edisilate that is equivalent to 10.0 mg of prochlorperazine base. Drug layer composition 2 in the tablet comprises 20 mg of prochlorperazine base. The total dose of drug within the tablet is equivalent to 30.0 mg of prochlorperazine base. A batch of these tablets is compressed.

The rate controlling membrane composition solution is next prepared by dissolving 40 grams A:B:A tri-block copolymer in 5,000 grams of acetone with warming and stirring. The tri-block copolymer is an ethylene oxide:propylene oxide ethylene:oxide tri-block copolymer having an average molecular weight ranging from 12,700 to 17,400. The number of ethylene oxide monomer units per block is approximately 141 and the number of propylene oxide monomer units per block is approximately 44. The tri-block copolymer is available from BASF Corporation as Lutrol F108. Then, 160 grams of cellulose acetate is dissolved with stirring into the blend. The cellulose acetate has a molecular weight of approximately 50,000 and is commercially available from Eastman Chemical Company as Type CA-398-30.

The batch of tri-layer tablets are charged into a pharmaceutical pan coater and the rate controlling membrane composition solution is spray coated onto the tablets in a current of warm air. The batch of tablets is coated until a thickness of 4 mils of rate controlling membrane is applied uniformly on each tablet. The resulting batch of tablets is laser drilled with a 35 mil diameter delivery port on the drug layer end of the tablet. Finally, the drilled batch of tablets are dried for three days in forced air humid air maintained at 45° C. and 45% relative humidity to remove residual coating solvent. These compositions and procedures complete the fabrication of the sequential oral osmotic delivery system.

When administered orally to a patient in need of anti-emetic therapy, water from the gastrointestinal tract is imbibed across the rate controlling membrane into the delivery system by osmosis. Each of the three tablet layers simultaneously hydrates. Drug layer composition 1 and drug layer composition 2 form low viscosity hydrogels while the push layer composition forms a high viscosity hydrogel. As the push layer expands, it slowly extrudes drug layer 1 and then slowly extrudes drug layer 2. The drug salt is therefore released first over a few hours. Then the drug in oil form is released second over a prolonged period of time.

The net effect of these mechanisms is to produce a sequential delivery pattern comprising a first water-soluble drug delivery pattern in the upper gastrointestinal tract followed by a second delivery pattern of water insoluble drug in the lower tract to provide a dosage form for once a day anti-emetic therapy.

### Example 5

### Metformin Drug Form

A controlled release dosage form delivering metformin according to U.S. Pat. No. 6,419,954 to Chu et al. (“Chu”) is prepared, using the disclosure relating to manufacture of tablets comprising non-randomly distributed active agents (beginning at Column 20, line 58). While Chu teaches a dosage form useful in the practice of this invention, Chu fails to teach the application of that dosage form to the making and using of a controlled release dosage form comprising a micronized base form of a drug; either (i) a pharmaceutically acceptable salt form of the drug or (ii) starting materials that are capable of reacting to form a pharmaceutically acceptable salt form of the drug; an upper gastrointestinal system pharmaceutically acceptable salt form releasing structure; and a colonic system micronized base form releasing structure. The application of the disclosure of Chu to the inventive controlled release dosage forms is now explored in more detail.

The active ingredients are metformin base, and the HCL salt of metformin. The colonic system base form releasing structure is prepared as a tablet core comprising micronized metformin base according to the processes disclosed, inter alia, at columns 12 and 18, and compressed into a core tablet according to the disclosure, particularly at column 20. The core tablet form of the colonic system micronized base form releasing structure is now coated with one or more layers that contain the HCl salt form of metformin, according to the disclosure found in Chu, particularly at column 21. Such layers serve as the upper gastrointestinal system pharmaceutically acceptable salt form releasing structure. The release rate of the structures can be optimized according to the teachings of Chu to make a dosage form according to the invention.

### Example 7

### Captopril Drug Form

A controlled release dosage form according to U.S. Pat. No. 5,391,381 to Wong et al. (“Wong”) is prepared, using the disclosure relating to Example II, except that Captopril is used instead of porcine somatotropin. While Wong teaches a dosage form useful in the practice of this invention, Wong fails to teach the application of that dosage form to the making and using of a controlled release dosage form comprising a micronized or liquid base form of a drug; either (i) a pharmaceutically acceptable salt form of the drug or (ii) starting materials that are capable of reacting to form a pharmaceutically acceptable salt form of the drug; an upper gastrointestinal system pharmaceutically acceptable salt form releasing structure; and a colonic system micronized base form releasing structure. The application of the disclosure of Wong to the inventive controlled release dosage forms is now explored in more detail.

The active ingredients are captopril base, and the HCL salt of captopril. The colonic system base form releasing structure is prepared as dispensing components of Wong according to the processes disclosed, inter alia, at column 18, with the exception that captopril base is substituted for porcine somatotropin. The upper gastrointestinal system pharmaceutically acceptable salt form releasing structure is now made according to the processes disclosed in Wong, inter alia, at column 18, with the exception that captopril HCl is substituted for porcine somatotropin. The delivery system subassembly is assembled such that the upper gastrointestinal system pharmaceutically acceptable salt form releasing structure will be discharged prior to the discharge of the colonic system base form releasing structure. The release rate of the structures can be optimized according to the teachings of Wong to make a dosage form according to the invention.

1. A controlled release dosage form comprising:
(a) a micronized or liquid base form of a drug;
(b) either (i) a pharmaceutically acceptable salt form of the drug or (ii) starting materials that are capable of reacting to form a pharmaceutically acceptable salt form of the drug;
(c) an upper gastrointestinal system pharmaceutically acceptable salt form releasing structure; and
(d) a colonic system base form releasing structure.
